(19) United States
(12) Patent Application Publication
(10) Pub. No.: US 2013/0084309 A1
(43) Pub. Date: Apr. 4, 2013

(54) PROCESS FOR THE PREPARATION OF O/W EMULSION

(71) Applicant: Joel Colonval, Rixensart (BE)

(72) Inventor: Joel Colonval, Rixensart (BE)

(73) Assignee: GLaxoSmithKline Biologicals S.A., Rizensart (BE)

(21) Appl. No.: 13/702,779

(22) PCT Filed: Jun. 8, 2011
(86) PCT No.: PCT/EP11/59487

§ 371 Date: Dec. 7, 2012

(30) Foreign Application Priority Data
Jun. 10, 2010 (GB) 1009671.7

(51) Int. Cl. A61K 9/107; A61K 39/39

(52) U.S. Cl. CPC A61K 9/1075; A61K 39/39
USPC 424/283.1

(57) ABSTRACT

The present invention provides a process for the production of an oil in water emulsion comprising the steps of: a) dissolving a surfactant in a volume of water or aqueous solution to produce a surfactant solution; and b) diluting the surfactant solution in a volume of water or aqueous solution greater than the volume of the surfactant solution to produce an aqueous phase.

### TECHNICAL FIELD

The present invention relates to improved processes for the production of oil in water emulsions, in particular, submicron oil in water emulsions comprising squalene.

### BACKGROUND TO THE INVENTION

The present invention relates to processes for the production of oil in water emulsions. Methods of manufacture are disclosed in Ott et al., 2000 (The Adjuvant MF59: A 10-year Perspective. Vaccine Adjuvants: Preparation methods and Research Protocols [Methods in Molecular medicine, Vol. 42, Chapter 12, p211-228], Ott et al., 1995 (MF59—Design and Evaluation of a Safe and Potent Adjuvant for Human Vaccines: Vaccine Design, the Subunit and Adjuvant Approach [Pharmaceutical Biotechnology volume 6] eds. Powell & Newman, WO06/100110A1 and Lidgate et al., 1992 (Sterile Filtration of a Parenteral Emulsion. Pharmaceuticals Research 9(7): 860-863).

Oil in water emulsions can be used in vaccine/immunogenic compositions as adjuvants. As these emulsions are administered to humans it is necessary that the emulsions are sterile. Oil in water emulsions used as adjuvants are submicron emulsions and the oil droplets are sufficiently small to be sterile-filtered through 0.2 μm filters. It is an object of the present invention to provide a process for the production of submicron oil in water emulsions.

### SUMMARY OF THE INVENTION

The present invention relates to a process for production of oil in water emulsions, in particular, a process for the production of an oil in water emulsion comprising the steps of: a) dissolving a surfactant in a volume of water or aqueous solution to produce a surfactant solution; and b) diluting the surfactant solution in a volume of water or aqueous solution greater than the volume of the surfactant solution to produce an aqueous phase.

### DETAILED DESCRIPTION OF THE INVENTION

Oil in water emulsions are made by combining and mixing an oil phase (comprising one or more oils and optionally one or more surfactants) with an aqueous phase comprising a surfactant. The surfactant allows a stable emulsion to be formed i.e. an emulsion that will not separate into oil and aqueous phases over a short period of time.

The present inventors have demonstrated that by dissolving a surfactant in a small volume of liquid before adding the resulting surfactant solution to a larger volume they ensure complete dissolution of the surfactant in the aqueous phase. If the surfactant is added immediately to a large volume of liquid, the surfactant collects at the bottom of the container comprising the large volume of liquid (for example, a tank) making it difficult to dissolve. If surfactant remains at the bottom of the tank, the composition and stability of the emulsion may be affected.

Accordingly, the present invention provides a process for the production of an oil in water emulsion comprising the steps of: a) dissolving a surfactant in a volume of water or aqueous solution to produce a surfactant solution; and b) diluting the surfactant solution in a volume of water or aqueous solution greater than the volume of the surfactant solution to produce an aqueous phase.

The processes of the present invention are particularly beneficial when producing large volumes of oil in water emulsion and thus using large volumes of aqueous phase. Accordingly, in a particular embodiment of the invention there is provided a process as described herein wherein the volume of oil in water emulsion produced is more than about, 10, 15, 20, 25, 30, 35, 40, 50, 60, 70, 80, 90, 100, 200, 250, 300, 350, 400, 450, 500 or 550 litres (L), for example 50 L, 280 L or 560 L scale.

The surfactant solution can be added to a volume of water or aqueous solution greater than the volume of the surfactant solution by any means, for example pouring. In a particular embodiment the surfactant solution is added to a volume of water or aqueous solution greater than the volume of the surfactant solution using a pump, in particular a peristaltic or membrane pump.

The processes of the present invention are particularly beneficial when using large volumes of aqueous phase and thus in one embodiment there is provided a process as described herein wherein the aqueous phase comprises between about 80% and about 98%, about 85% and about 97%, 89% and 96% or about 90% to 95% for example about 90% or about 95% (v/v) of the oil in water emulsion.

The volume of the surfactant solution [i.e. the product of step a)] in the present invention is less than the volume of water or aqueous solution in which it is diluted in step b).

Accordingly, in one embodiment the surfactant solution comprises about 0.001% to about 40%, about 0.01% to about 20%, about 0.02% to about 15%, about 0.03% to about 10%, about 0.04 to about 5%, for example 0.1, 0.2, 0.3, 0.4, 0.5, 0.6, 0.7, 0.8, 0.9, 1, 2 ,3, 4, 5, 6, 7, 8, 9, or 10% (v/v) of the total volume of the aqueous phase.

The surfactant is dissolved in either water or an aqueous solution. In particular the water may be water for injection (WFI), i.e. water that is suitable for parenteral use.

Alternatively, the surfactant is dissolved in an aqueous solution such as a buffer. The buffer is also suitable for parenteral use.

Suitable buffers are well known to the person skilled in the art and include but are not limited to a phosphate buffer, citrate buffer, Tris buffer, succinate buffer, maleate buffer or borate buffer. In a particular embodiment, the buffer is selected from the group, phosphate buffered saline (PBS), modified PBS (PBS-mod) and citrate buffer.

If a buffer is used in step a) then in a particular embodiment, the buffer may be concentrated compared to the final concentration in the aqueous phase, for example, the buffer may be about 5 to 25 times concentrated e.g. 5, 10, 15, 20, 25, 30 times concentrated. If water is used in step a) then in a particular embodiment a buffer is used to dilute the surfactant solution, the buffer may or may not be concentrated.

In a particular embodiment of the invention there is provided a process for the production of an oil in water emulsion comprising the steps of: a) dissolving a surfactant polyoxyethylene sorbitan monooleate) in a volume of concentrated buffer (e.g. PBS-mod) to produce a surfactant solution; and b) diluting the surfactant solution in a volume of water for injection (WFI) greater than the volume for the surfactant solution to produce an aqueous phase.

In a particular embodiment of the invention there is provided a process for the production of an oil in water emulsion comprising the steps of: a) dissolving a surfactant (e.g. polyoxyethylene sorbitan monooleate) in a volume of WFI to produce a surfactant solution; and b) diluting the surfactant solution in a volume of buffer (e.g. citrate buffer), optionally concentrated buffer, greater than the volume for the surfactant solution to produce an aqueous phase.

In one embodiment, a buffer may be used in both step a) and b), and in a particular embodiment, the same buffer is used in both steps a) and b). In such circumstances the buffer is typically not concentrated; however, in one embodiment the buffer in step a) is concentrated whereas as the buffer is step b) is a diluted buffer.

Suitable surfactants are well known to the skilled person and include, but are not limited to polyoxyethylene sorbitan monooleate (TWEEN 80, POLYSORBATE 80), sorbitan triolate (SPAN 85), phosphatidylcholine (lecithin), and octoxynol-9 (TRITON X-100). In a particular embodiment of the invention the surfactant used in step a) is polyoxyethylene sorbitan monooleate (TWEEN 80, POLYSORBATE 80).

In an embodiment of the invention, the amount of surfactant to be mixed into the aqueous phase is that amount to give a final concentration between about 0.1 and about 1.5%, for example about 0.5% and about 1% (w/v) of said surfactant in the oil in water emulsion. It should be noted that one or more other surfactants may be added to the oil phase, for example sorbitan trioleate, maybe added to the oil phase (comprising squalene for example) before mixing the oil and aqueous phases together.

In a further embodiment of the invention there is provided a process as described herein further comprising the step c) introducing the aqueous phase [i.e. the product of step b)] into a mixing device.

The term “mixing device” as used herein means a device suitable for mixing an oil phase and an aqueous phase to form an emulsion. In a particular embodiment of the invention the mixing device is a high shear mixing device. Suitable high shear mixing devices are known to the skilled person and include, but are not limited to a high-speed blade homogeniser, an inline homogeniser, a colloid mill or a sonolator. In a particular embodiment of the invention the mixing device is a high-pressure homogeniser. Suitable high pressure homogenizers are known to the skilled person and include, but are not limited to a fixed geometry microfluidiser or to a variable geometry high pressure homogenizer.

In a further embodiment of the invention there is provided a process as described herein further comprising the step of d) introducing an oil phase into a mixing device as described herein.

In a particular embodiment of the invention the introduction of the aqueous phase [step c)] and the oil phase [step d)] are performed substantially simultaneously.

In a particular embodiment steps c) and or d) are performed by applying a positive pressure in the oil phase and/or aqueous phase containing tank.

The oil phase may comprise one or more metabolisable oils. In a particular embodiment, the oil phase comprises squalene or squalane, in particular squalene. In a further embodiment of the invention the oil phase comprises a tocol and in a particular embodiment of the invention the oil phase comprises α-tocopherol. In a further embodiment of the invention the oil phase comprises squalene and α-tocopherol.

The oil phase may further comprise a surfactant as described herein. In a particular embodiment of the invention the oil phase comprises sorbitan triolate (SPAN 85) and in a particular embodiment of the invention, the oil phase comprises squalene and sorbitan triolate (SPAN 85).

In a further embodiment of the invention the aqueous and oil phases as described herein are introduced at a ratio of about 90:10 or about 95:5 (percent v/v).

In a further embodiment of the invention there is provided a process as described herein further comprising the step e) mixing the oil and aqueous phases to form an oil in water emulsion.

Following mixing in step e) the oil in water emulsion may be a coarse oil in water emulsion if mixed in a high shear mixing device for example. In order to reduce the size of the oil droplets in the oil in water emulsion so that it is suitable for sterile filtration for example, the emulsion from step e) can be further processed in for example a high-pressure homogeniser.

Accordingly, in a further embodiment of the invention there is a process as described herein further comprising the step f) subjecting the oil in water emulsion of step e) to high pressure homogenization to form a submicron oil in water emulsion.

The skilled person can achieve the desired oil droplet size by varying the number of time the emulsion is passed through the high pressure homogeniser, as the oil droplet size will reduce after each cycle. Accordingly, in a one embodiment of the invention there is provided a process as described herein where in the emulsion is subjected to high pressure homogenisation [step f)] 1, 2, 3, 4, 5, 6, 7, 8 or more times.

In a further embodiment of the invention the high pressure homogenization is performed at a pressure of between about 10000 and about 20000, about 12000 and about 18000, about 14000 and about 16000 or about 15000±1000 psi.

During high pressure homogenisation the temperature of the oil in water emulsion typically increases and thus in a one embodiment of the invention the oil in water emulsion is cooled to between about 15° C. and about 30° C., about 16° C. and about 29° C., about 17° C. and about 28° C., about 16° C. and about 27° C. or between about 16 ° C. and about 28° C. after the one or more, but at least the final time the emulsion is subjected to high pressure homogenisation.

The terms “comprising”, “comprise” and “comprises” herein are intended by the inventors to be optionally substitutable with the terms “consisting of”, “consist of” and “consists of”, respectively, in every instance.

The word “substantially” does not exclude “completely” e.g. a composition which is “substantially free” from Y may be completely free from Y. Where necessary, the word “substantially” may be omitted from the definition of the invention.

The term “about” in relation to a numerical value x means x±5 or 10%.

The invention will now be described further by way of reference to the following, non-limiting examples.

### EXAMPLES

### 1.1 Aqueous Phase Preparation

Stainless steel tanks were used for the production of the aqueous phase (referred to as tank 1), and for the emulsion circuit (referred to as tank 2) were inerted with nitrogen (N₂) before use.

The aqueous phase was prepared in two steps:
a. Polysorbate 80 was dissolved in a concentrated isotonic phosphate-buffered saline solution (PBS-mod buffer 20×[see Table 1 below]) contained in a bottle or a plastic bag.
b. The water for injection (WFI) was filled in a stainless steel tank (tank 1), and N₂ inerted before use. For small scale processes (up to 70 L), the PBS-mod buffer 20 x/polysorbate 80 mix was then poured into the tank through opening in the top plate, diluted in the WFI contained in tank 1 and stirred until homogeneity was achieved. Alternatively, at larger scale (over 70 L), polysorbate 80 diluted in phosphate buffer saline solution was further diluted into about 5 L of water taken into tank 1, and then the diluted solution was pumped into tank 1 with a peristaltic pump. In this case the dilution in WFI was necessary to obtain a less viscous solution that it was possible to pump.

TABLE 1
PBS-mod buffer (20x concentrated)

| | Buffer 20x concentrated | | | |
|---|---|---|---|---|
| NaCl | 2.74 | M | 160 | g/L |
| KCl | 53.6 | mM | 4 | g/L |
| Na₂HPO₄ | 162 | mM | 23 | g/L |
| KH₂PO₂ | 29.4 | mM | 4 | g/L |

1.2 Oil phase preparation

The oil phase was prepared by adding D,L-α-tocopherol to squalene contained in a stainless steel oil tank. The quantities of each component depend on the final volume of emulsion to produce, and are determined by weight. The mix was stirred until homogeneity was achieved. The mix was flushed with N₂.

### 1.3 Emulsification

Emulsification was performed at room temperature.

The aqueous and oil phases were simultaneously introduced in a high shear homogenizer with an injection ratio of 10% oil phase/90% aqueous phase (v/v).

After the high shear homogenizer, the emulsion was directly introduced into a high pressure homogenizer to reduce the droplet size. A nitrogen flush was maintained in the oil tank during the 1st pass, and in tank 1 and tank 2 throughout the entire emulsification process.

After the 1st pass, the resulting emulsion was collected in an apyrogenous stainless steel tank (tank 2).

When the entire volume of the batch passed through the high pressure homogenizer, the batch was processed a second time through the same equipment, passing from tank 2 to tank 1 (2nd pass).

The batch proceeded through the high pressure homogenizer for a third pass, and was collected in tank 2 (3rd pass).

The emulsion contained in a tank was inerted by N₂ flushing on the headspace and stored under N₂ pressure before filtration.

1. A process for the production of an oil in water emulsion comprising the steps of: a) dissolving a surfactant in a volume of water or aqueous solution to produce a surfactant solution; and b) diluting the surfactant solution in a volume of water or aqueous solution greater than the volume of the surfactant solution to produce an aqueous phase, wherein the volume of oil in water emulsion to be produced is more than about 25 to 550 litres (L) scale.

2. The process of claim 1 wherein the volume of surfactant solution comprises about 0.001% to about 40%, (v/v) of the total volume of the aqueous phase.

3. (canceled)

4. The process of claim 1 wherein the surfactant solution is added to a volume of water or aqueous solution greater than the volume of the surfactant solution using a pump.

5. The process of claim 1 wherein the aqueous phase comprises between about 80% and about 98%, (v/v) of the oil in water emulsion.

6. The process of claim 1 wherein the surfactant is polyoxyethylene sorbitan monooleate.

7. The process of claim 1 wherein the aqueous solution of step a) comprises a buffer.

8. The process of claim 7 wherein water is used in step b).

9. The process of claim 1 wherein water is used in step a).

10. The process of claim 9 wherein the aqueous solution of step b) comprises a buffer.

11. The process of claim 1 wherein an aqueous solution comprising a buffer is used in both step a) and step b).

12. The process of claim 6 wherein the buffer comprises a sodium citrate—citric acid buffer or is a phosphate-buffered saline (PBS) solution.

13. (canceled)

14. (canceled)

15. The process of claim 1 further comprising
step c) introducing the aqueous phase into a mixing device or
step d) introducing an oil phase into a mixing device; or
both step c) and step d).

16. (canceled)

17. (canceled)

18. The process according to claim 12 wherein step c) is performed while applying a positive pressure in the oil phase mixing device; or
step d) is performed while applying positive pressure in the aqueous phase mixing device; or
steps c) and d) are performed while applying a positive pressure in the oil phase and aqueous phase containing mixing devices.

19. (canceled)

20. (canceled)

21. The process of claim 12 comprising the step e) mixing the oil and aqueous phases to form an oil in water emulsion.

22. The process of claim 21 further comprising the step of f) subjecting the oil in water emulsion of step e) to high pressure homogenization to form a submicron oil in water emulsion.

23-30. (canceled)
